(19) United States
(12) Patent Application Publication
(10) Pub. No.: US 2025/0161018 A1
(43) Pub. Date: May 22, 2025

(54) SYSTEMS, DEVICES, AND METHODS FOR BONE SUTURE ATTACHMENT AND SUPPORT

(71) Applicant: AESCLEPIUS CORPORATION, La Jolla, CA (US)

(72) Inventors: Adam H. Hsieh, La Jolla, CA (US); Joe Ty Lin, San Diego, CA (US)

(21) Appl. No.: 18/926,462

(22) Filed: Oct. 25, 2024

Related U.S. Application Data
(63) Continuation of application No. PCT/US23/19909, filed on Apr. 26, 2023.
(60) Provisional application No. 63/335,404, filed on Apr. 27, 2022.

(51) Int. Cl. A61F 2/08; A61B 17/04

(52) U.S. Cl. CPC A61F 2/0811; A61B 17/0401; A61B 2017/0414; A61B 2017/0427; A61B 2017/0458; A61F 2002/0817; A61F 2002/0852; A61F 2002/0864; A61F 2002/0882; A61F 2210/0076; A61F 2230/0091

(57) ABSTRACT

Systems, devices, and methods are provided for attaching and supporting a bone suture. In particular, described herein are embodiments of implantable bracing apparatuses comprising a cylindrical volume comprising one or more helices, at least a portion of which is configured to be implanted within a bone tunnel. In some embodiments, the implantable bracing apparatus can further comprise an outer portion of the cylindrical volume configured to interface with the bone tunnel, an inner portion configured to pass one or more sutures therethrough, a first open end corresponding with a first entry of the bone tunnel, and a second open end corresponding with a second entry point of the bone tunnel.

### CROSS-REFERENCE TO RELATED APPLICATIONS

This application is a continuation of International Patent Application No. PCT/US2023/019909, filed Apr. 26, 2023, which claims priority to and the benefit of U.S. Provisional Patent Application No. 63/335,404 filed Apr. 27, 2022, both of which are incorporated by reference herein in their entireties for all purposes.

### FIELD

The subject matter described herein relates generally to systems, devices, and methods for attaching and supporting a suture to a bone. In particular, described herein are embodiments of bracing apparatuses for bone suture attachment and support, as well as methods and devices relating thereto.

### BACKGROUND

Joint arthropathies (diseases that compromise joint function) are part of a steadily growing worldwide trend in chronic musculoskeletal disorders. In 2012, the Bone and Joint Initiative published findings that one out of every two Americans were diagnosed with musculoskeletal conditions, accounting for hundreds of billions of dollars in costs, which continue to grow annually. In 2018, the World Health Organization (WHO) identified the second largest contributor to global disability as musculoskeletal conditions. The increasing number of afflicted people and a continued rise in treatment costs point to a critical need for new technologies that provide more effective solutions to manage musculoskeletal ailments.

Joint arthropathies caused by soft tissue damage (e.g., tendon, ligament, and/or fibrocartilage tears) make up the majority of cases within the broader category of musculoskeletal conditions. Shoulder pain stands among the most common musculoskeletal complaint worldwide, with rotator cuff tears being the leading cause of shoulder disability. Other types of ligament, tendon, and fibrocartilage injuries, such as labral tears, meniscus root tears, Achilles tendon avulsions, anterior cruciate ligament (ACL) ruptures, and lateral ankle ligament tears, among others, are somewhat less prevalent, but no less debilitating. Most of these injuries, whether due to tear size or lack of responsiveness to conservative treatment (e.g. physical therapy), require primary surgical repair. In 2014, the United States Agency for Healthcare Research and Quality (AHRQ) reported over 1.8 million invasive, therapeutic surgeries involving “muscle, tendon, soft tissue operating room procedures” and “incision or fusion of joint, or destruction of joint lesion” in the United States, which equates to 8.3% of the roughly 21.7 million total ambulatory and inpatient surgical procedures.

The goal of such repairs is to re-establish the position and direction of force transmission in these tissues, in order to restore stability and motion to their respective joints. For soft tissue injuries, this can be achieved by re-attaching the torn areas of soft tissue (e.g., tendon, ligament, and/or fibrocartilage)—which naturally pulls away from its anatomic insertion site upon injury-using a fixation method to create a stable connection and close contact between tissue and bone so that the interface can heal over time. The fixation method should be mechanically and structurally robust, because the biomechanical forces generated by muscles and joint motion may reach several hundred Newtons during physiological function. The fixation method should also be sufficient to withstand thousands of cycles of repetitive loading, particularly in the lower extremities.

Due to anatomical and functional variation, techniques used to achieve soft tissue repair can depend on the particular application. Two approaches for soft tissue reattachment, for example, include: (1) transosseous repair and (2) suture anchored repairs.

In a transosseous approach, a bone tunnel is created at a location corresponding to the injured tissue. A remaining portion of the torn tissue, a synthetic/autologous/cadaveric tissue graft, or a suture tethered to the injured tissue can be passed through the bone tunnel and either tied back around the surface of the bone or affixed by an interference screw or button device. This approach is traditionally performed as open surgery, which may require a large incision to give the surgeon access to the bone and joint, or more recently with arthroscopic surgical tools.

Due to anatomical limitations, a transosseous approach for rotator cuff repairs involves passing sutures through multiple tunnels that intersect at a location within bone. At this intersection, angular features place sutures at risk for abrasive failure. Moreover, even in the absence of sharp angles, sutures can cut through bone unpredictably, for example, when the sutures are being tensioned during surgery. In certain cases, a surgeon can insert a cortical reinforcement, such as a polymer grommet, into the entrance of the bone tunnel to reduce failure risk. However, this method can be disadvantageous for various reasons, including requiring the subjective judgment of the surgeon, and can result in a high variability in outcomes. Moreover, there are constraints in implementing multiple bone tunnels because of the long traverse required.

Suture anchored repair is another approach to repair rotator cuff tendons, as well as the labrum, meniscus root, lateral ankle ligaments, and others. With respect to suture anchored repair, a metal or polymer suture anchor is secured by way of screw or interference fit into a pilot hole created in the bone. Sutures are tethered to the anchor and are used to tie the tissue back to its anatomic insertion site, thereby restoring function. In some cases, the anchor can include suture knots and/or deployable securing elements. In other cases, the anchor can be an all-suture soft anchor comprising a polymer textile sleeve through which a suture runs. Once inserted into the pilot hole, the sleeve bunches together when the suture line is pulled, creating a plug that is slightly wider than the pilot hole, to hold the suture in place.

Suture anchored repair relies on the interface between the anchor and the bone to maintain structural integrity of the repair. This can be disadvantageous in that the anchors lack the ability to achieve full biological integration, such that with time there will be a risk of failure if tissue healing remains inadequate. Even with a secure suture anchor, the rate of re-tear in a rotator cuff repair has been reported to be between 30% and 70%. Furthermore, the size and placement of anchors limit the sutures that can be used for repair. For instance, if there are complications or failures in a primary repair where a suture anchor is used, surgeons are faced with the dilemma of having constraints on anchor placement for the secondary repair.

Thus, needs exist for systems, devices and methods that are more mechanically and structurally robust for attaching and supporting a bone suture.

### SUMMARY

Provided herein are example embodiments of systems, devices and methods for attaching and supporting a suture to a bone. Generally, an implantable bracing apparatus comprising a cylindrical volume is described, wherein the cylindrical volume comprises one or more helices at least a portion of which is configured to be implanted within a bone tunnel.

In some example embodiments, for example, the implantable bracing apparatus can comprise an outer portion of the cylindrical volume that is configured to interface with the bone tunnel, an inner portion configured to pass one or more sutures therethrough and to prevent the one or more sutures from contacting at least a portion of the bone tunnel, a first open end corresponding with a first entry point of the bone tunnel, and a second open end corresponding with a second entry point of the bone tunnel.

In some of the example embodiments, the implantable bracing apparatus can comprise a single helical coil.

The various configurations of these systems, methods and devices are described by way of the embodiments which are only examples. Other systems, devices, methods, features, improvements and advantages of the subject matter described herein are or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are a side view and a perspective view of an example embodiment of an implantable bracing apparatus.

FIGS. 2A and 2B are a side view and a perspective view of another example embodiment of an implantable bracing apparatus.

FIG. 3 is a perspective view of another example embodiment of an implantable bracing apparatuses.

FIGS. 4A and 4B are a side view and a perspective view of another example embodiment of an implantable bracing apparatus.

FIG. 5 is a side view of another example embodiment of an implantable bracing apparatus.

FIG. 6 is a perspective view of another example embodiment of an implantable bracing apparatus.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for attaching and supporting a bone suture. Accordingly, some embodiments include implantable bracing apparatuses to reinforce a hole or tunnel in a bone. These various embodiments can include elements through which sutures pass and/or to which sutures may be tethered. In certain embodiments, some or all elements of the bracing apparatus may comprise metal, natural or synthetic material, organic or inorganic material, biodegradable or non-biodegradable polymer, or a combination thereof.

In some embodiments, a bracing apparatus can comprise a cylindrical volume, further comprising one or more helices, that is inserted into a pre-formed bone tunnel. The bracing apparatus can further comprise ends having one or more features to provide specific functionality.

In some embodiments, for example, a bracing apparatus can comprise a coil having single or multiple helices, wherein the single or multiple helices are configured to enhance flexibility, deformability, and porosity of the bracing apparatus.

### Example Embodiments of Implantable Bracing Apparatuses

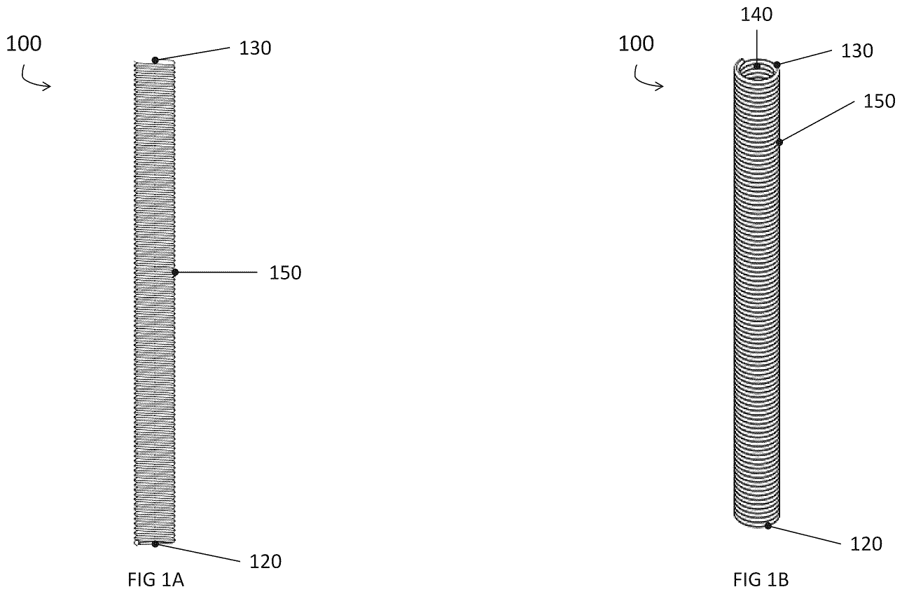

FIGS. 1A and 1B depict a side view and a perspective view, respectively, of an example embodiment of an implantable bracing apparatus 100. According to some embodiments, a bracing apparatus can be manufactured as a single helical coil that encompasses a cylindrical volume. Although FIGS. 1A and 1B show a right-hand close wound helical coil, those of skill in the art will appreciate that the helical coil can be open wound, and can possess any combination of different chirality, pitch, radius of curvature, slant angle, wire diameter, wire material, and wire cross-sectional geometry. Furthermore, helical coil bracing apparatuses can also possess spatial variations of different parameters within a single embodiment, and can possess cross-sectional geometries of any shape, circular or non-circular. According to one aspect of the embodiments, outer surface 150 is configured to interface with the bone tunnel, and inner lumen 140 is configured to pass a suture therethrough and to prevent the suture from contacting at least a portion of the bone tunnel. According to another aspect of the embodiments, first open end 120 of apparatus 100 can correspond with a first entry point of the bone tunnel, and second open end 130 can correspond with a second entry point of the bone tunnel.

Furthermore, some or all elements of bracing apparatus 100 can comprise a metallic material, natural or synthetic material, organic or inorganic material, biodegradable or non-biodegradable polymer, or a combination thereof. According to certain embodiments, for example, inner lumen 140 can comprise a coating of polyethylene or polytetrafluoroethylene composites to reduce friction. According to other embodiments, osteoconductive materials, such as hydroxyapatite, can be used to coat inner lumen 140 and/or outer surface 150 to enhance osseointegration and/or bone ingrowth. Proteins, other biologics, or synthetic molecules can also be attached to either inner lumen 140 or outer surface 150 to achieve the same or similar results. Further, some or all elements of bracing apparatus 100 can be subject to surface modification to enhance osseointegration, such as, for example, plasma treatment or electrochemical etching to generate nanotextured surfaces.

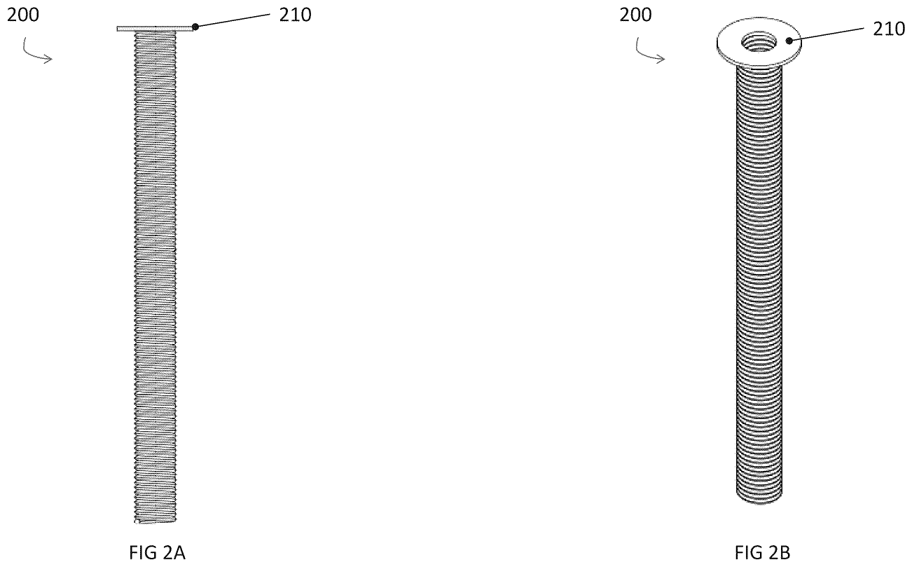

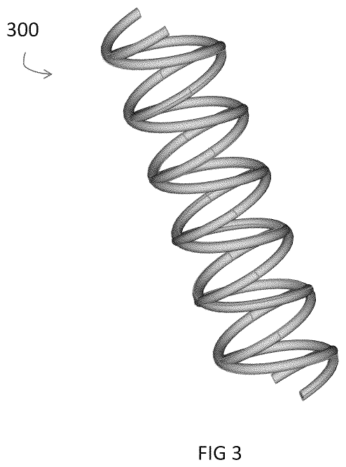

FIGS. 2A and 2B depict a side view and a perspective view, respectively, of another example embodiment of an implantable spring bracing apparatus 200. According to some embodiments, a bracing apparatus can have features on one or both ends of the helical coil to provide specific functionalities. The example embodiment shown in FIGS. 2A and 2B comprises a single helical coil and a flange 210 on one end of the helical coil. Flange 210 is shown as a solid circular flat disk with a centered through hole opening that is coincident with the lumen of the helical coil, and is continuous with the helical coil. However, those of skill in the art will appreciate that flange 210 can be of any regular or irregular three-dimensional geometry, can be uniform or non-uniform in thickness, possess holes or spaces, can have a through hole opening that is centered or off-centered on the flange, can have an opening that is smaller than the lumen of the helical coil, or can have no through hole opening thereby completely closing off the lumen of the helical coil. Additionally, flange 210 can include other features including, but not limited to, loops, arches, or hooks for anchoring sutures, spikes on the surface of the flange that interfaces with bone, or any organic molecular coating, inorganic molecular coating, or surface modification to enhance osseointegration. Those of skill in the art will also appreciate that flange 210 can be formed continuously with the material of the helical coil, or can be directly affixed to the helical coil by welding, mechanical interlock, or any other means of secure attachment.

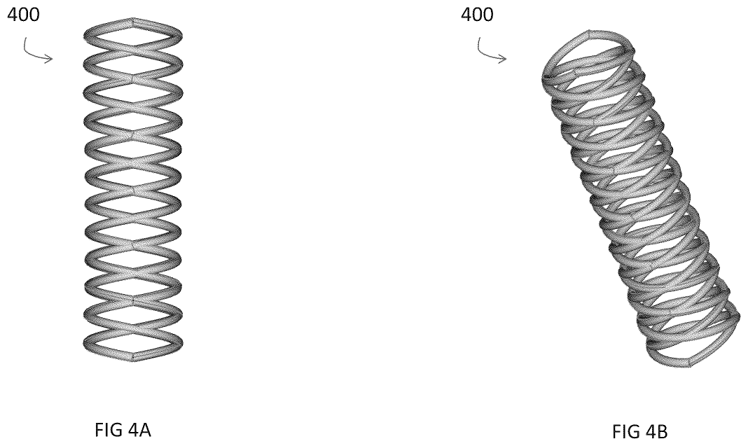

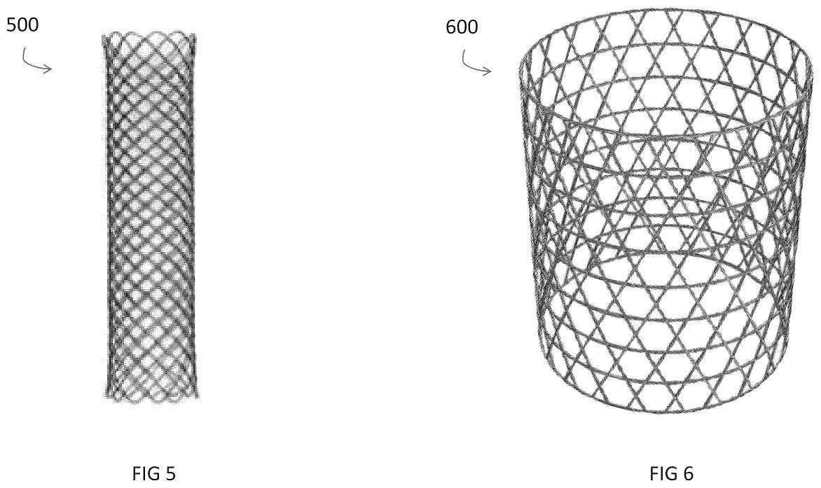

FIG. 3 is a perspective view of an embodiment of implantable bracing apparatus 300 comprising a cylindrical volume comprising a double helix, which—in a manner similar to that of apparatus 100—can have different physical and material characteristics, as well as spatial variations. FIGS. 4A and 4B depict a side view and a perspective view, respectively, of another example embodiment of implantable bracing apparatus 400, wherein bracing apparatus 400 comprises a cylindrical volume comprising four helical coils (e.g., two right-hand and two left-hand coils). According to certain embodiments, bracing apparatuses can also comprise a cylindrical volume comprising a lattice of geometric cells formed by struts, such as those depicted in FIGS. 5 and 6. FIG. 5 shows a side view of an example embodiment of an implantable bracing apparatus 500 comprising a lattice of quadrilateral cells. FIG. 6 shows a perspective view of an example embodiment of an implantable bracing apparatus 600 comprising a lattice of multiple geometries (e.g., triangular and hexagonal). These embodiments of the latticework are intended to be illustrative only and are not meant to limit the scope of the present disclosure. Lattices can also comprise cells having any other geometries, regular or irregular, uniform and non-uniform, and having open or closed cell structures. Those of skill in the art will understand that, similar to bracing apparatuses 100 and 200, any of the example embodiments shown in FIGS. 3-6 can comprise a metallic material, natural or synthetic material, organic or inorganic material, biodegradable or non-biodegradable polymer, or a combination thereof. Those of skill in the art will also appreciate that any molecule or substance, whether organic or inorganic, natural or synthetic, can be used to coat the inner lumen and/or outer surface, and any surface modification can be performed on these same example embodiments to enhance osseointegration. Those of skill in the art will also appreciate that any of the example embodiments shown in FIGS. 3-6 can comprise features on one or both ends of the helical coil to provide specific functionalities including, but not limited to, a flange having any combination of features, as described for implantable spring bracing apparatus 200.

Additional example embodiments of implantable bracing apparatuses are described in U.S. patent application Ser. No. 17/570,039, which is incorporated in its entirety for all purposes.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. An implantable bracing apparatus for supporting one or more sutures, the implantable bracing apparatus comprising a cylindrical volume comprising one or more helices at least a portion of which is configured to be implanted within a bone tunnel, the cylindrical volume comprising:
an outer portion of the cylindrical volume configured to interface with the bone tunnel;
an inner portion of the cylindrical volume configured to pass the one or more sutures therethrough and to prevent the one or more sutures from contacting at least a portion of the bone tunnel;
a first open end corresponding with a first entry point of the bone tunnel;
a second open end corresponding with a second entry point of the bone tunnel; and
at least one flanged head disposed on at least one of the first open end and the second open end.

2. The implantable bracing apparatus of claim 1, wherein the cylindrical volume further comprises a single helical coil.

3. The implantable bracing apparatus of claim 2, wherein the cylindrical volume has a circular cross-section.

4. The implantable bracing apparatus of claim 2, wherein the cylindrical volume has a non-circular cross section.

5. The implantable bracing apparatus of claim 2, wherein the helical coil comprises a metallic material, a natural material, a synthetic material, an organic material, an inorganic material, a biodegradable polymer, a non-biodegradable polymer, or a combination thereof.

6. The implantable bracing apparatus of claim 2, wherein an inner lumen of the helical coil comprises a coating configured to reduce friction.

7. The implantable bracing apparatus of claim 6, wherein the coating is a polyethylene or polytetrafluoroethylene composite.

8. The implantable bracing apparatus of claim 2, wherein one or both of an inner lumen of the helical coil and the outer portion comprise at least one of an osteoconductive material, a protein, a biologic, or a synthetic molecule configured to promote osseointegration and bone ingrowth.

9. The implantable bracing apparatus of claim 8, wherein the osteoconductive material comprises hydroxyapatite.

10. The implantable bracing apparatus of claim 2, wherein at least a portion of the helical coil comprises a nanotextured surface.

11. (canceled)

12. The implantable bracing apparatus of claim 1, wherein the at least one flanged head includes a through hole opening configured to enable passage of the one or more sutures

13. The implantable bracing apparatus of claim 2, wherein the at least one flanged head comprises one or more of the following characteristics: an irregular three-dimensional geometry, a non-uniform thickness, a plurality of pores or spaces, and an off-set positioning relative to the helical coil.

14. The implantable bracing apparatus of claim 1, wherein the at least one flanged head includes one or more loops, arches, or hooks, wherein the one or more loops, arches, or hooks are configured for anchoring sutures.

15. The implantable bracing apparatus of claim 1, wherein the at least one flanged head includes a plurality of spikes on the surface of the flange, wherein the plurality of spikes is configured to interface with bone.

16. The implantable bracing apparatus of claim 1, wherein the at least one flanged head comprises any organic molecular coating, inorganic molecular coating, or surface modification to enhance osseointegration.

17. The implantable bracing apparatus of claim 2, wherein a diameter of the through hole opening is smaller than a diameter of a lumen of the helical coil.

18-21. (canceled)

22. The implantable bracing apparatus of claim 2, wherein the at least one flanged head has no through hole opening and completely closes off a lumen of the helical coil.

23-26. (canceled)

27. The implantable bracing apparatus of claim 1, wherein the cylindrical volume further comprises a double helix.

28. The implantable bracing apparatus of claim 1, wherein the cylindrical volume further comprises four helical coils.

29-42. (canceled)
